(19) United States
(12) Patent Application Publication
(10) Pub. No.: US 2002/0193347 A1
(43) Pub. Date: Dec. 19, 2002

(54) N-substituted norephedrine chiral derivatives, their preparation and their use for the synthesis of optically active functionalised compounds by hydrogen transfer

(76) Inventors: Michel Bulliard, Angers (FR); Jean-Francois Carpentier, Baisieux (FR); Andre Mortreux, Hem (FR); Kathelyne Everaere, Lille (FR)

Correspondence Address: SCHNADER HARRISON SEGAL & LEWIS, LLP, 1600 MARKET STREET, SUITE 3600, PHILADELPHIA, PA, 19103

(21) Appl. No.: 10/059,652

(22) Filed: Jan. 29, 2002

Related U.S. Application Data
(63) Continuation of application No. PCT/FR00/02161, filed on Jul. 27, 2000.

(30) Foreign Application Priority Data
Jul. 29, 1999 (FR) 99/09864

(51) Int. Cl. A61K 31/695; A61K 31/137; C07F 7/04

(52) U.S. Cl. 514/63; 514/649; 514/651; 564/349; 556/450

(57) ABSTRACT

A ligand adapted for use in a process for enantioselective reduction of unsaturated compounds carrying functional groups by a hydrogen transfer method including an optically active N-substituted chiral derivative of norephedrine and the associated process.

The object of the present invention is N-substituted chiral derivatives of norephedrine and their use as ligands for the reduction by hydrogen transfer of carbonyl derivatives. The invention thus also pertains to the processes for enantioselective reduction of optically active carbonyl derivatives by the hydrogen transfer method.

The synthesis of optically active functionalized alcohols such as, e.g., the pyridinyl-1-ethanols, the hydroxyethers, the β-hydroxesters, the β,δ-dihydroxyesters, constitutes at present an important competitive industrial sector. Therefore, there is a need for catalytic systems for the synthesis of these alcohols which are increasingly more competitive in terms of costs and efficacy. Research efforts have notably been directed to finding new catalytic complex ligands based on ruthenium, iridium or rhodium which can lead to improved efficacy in terms of catalytic activity and enantioselectivity.

Catalytic systems are known which associate a ruthenium complex of the type [RuCl₂(arene)]₂ (arene=benzene, para-cymene, mesitylene, hexamethylbenzene) with an enantiomerically pure organic ligand such as an amino alcohol like (1R,2S)- or (1S,2R)-ephedrine, which will be designated below as compound “A”, (φ-ephedrine, 2-amino-1,2-diphenylethanols or an N-monotosyl-diamine such as (1S,2S)—ArSO₂NHCH(Ph)CH(Ph)NH₂ (Ar=4-CH₃C₆H₅: (1S,2S)-TsDPEN) which will be designated below as compound “B” (R. Noyori et al., Acc. Chem. Res. 1997, 30, 97-102; R. Noyori et al., J. Am. Chem. Soc. 1997, 119, 8738; PCT patent application WO 97/20789) or a bis(oxazoline)amine (X. Zhang et al., J. Am. Chem. Soc. 1998, 120, 3917). When these catalytic systems are brought into the presence of a base such as i-PrOK and a hydrogen donor such as isopropanol or formic acid, they enable the transformation of certain nonfunctional simple ketones such as the arylalkylketones, especially acetophenone, into the corresponding chiral alcohols.

On the other hand, a number of carbonyl compounds exist for which the aforementioned catalytic systems are not satisfactory because they are inactive or only slightly active and/or not enantioselective or only slightly enantioselective. This is the case in particular for certain functional ketones such as the aliphatic β-ketoesters. Thus, the reduction of ethyl acetoacetate by the catalytic system combining [RuCl₂(para-cymene)]2 with a ferrocenic chiral diamine takes place slowly in isopropanol at 80° C. to yield ethyl 3-hydroxybutyrate with a conversion of 92% and an enantiomeric excess of 20% (P. Knochel et al., Tetrahedron: Asymmetry 1998, 9, 1143). Similarly, although the Ru-[(1S,2S)-TsDPEN] system catalyzes the reduction of arylic β-ketoesters, PhCOCH₂COOR (R=alkyl), in formic acid to yield the corresponding β-hydroxyesters quantitatively with an enantiomeric excess between 75 and 95% (R. Noyori et al., Acc. Chem. Res. 1997, 30, 97-102; R. Noyori et al., J. Am. Chem. Soc. 1997, 119, 8738; PCT patent application WO 97/20789), examples presented in the experimental part below demonstrate that this system is ineffective in numerous cases,

According to the present invention, the N-substituted chiral derivatives of norephedrine constitute new amino-alcohol type ligands, which are simple to prepare and high performing in terms of activity and enantioselectivity for the synthesis of functional chiral alcohols by reduction of carbonyl derivatives by the hydrogen transfer method. This method, with which the expert in the field is familiar, is described, for example, in European patent application no. 916 637.

The optically active N-substituted chiral derivatives of norephedrine which can be used as ligands according to the invention correspond to formula (I) below

(I)

in which:

R represents a C₁₋₁₀ alkyl group, a saturated or unsaturated C₃₋₉ cycloalkyl group, an aryl group, said groups comprising possibly one or more substituents chosen from among a halogen atom such as chlorine, fluorine or bromine, an —NO₂ group, a C₁₋₅ alkyl, a C₁₋₅ alkoxy, a fused or unfused C₁₋₇ cycloalkyl, a fused or unfused aryl group, possibly substituted by a C₁₋₅ alkyl, a C₁₋₅ alkoxy, a halogen, said C₁₋₁₀ alkyl group, saturated or unsaturated C₃₋₉ cycloalkyl group or aryl group comprising possibly one or more heteroatoms such as O, N or Si.

R1 represents a hydrogen atom, a C₁₋₁₀ alkyl group such as methyl, ethyl, propyl or isopropyl, an aryl group such as a phenyl, a saturated or unsaturated C₃₋₉ cycloalkyl group, said groups comprising possibly one or more substituents selected from among a halogen atom such as chlorine, fluorine or bromine, an —NO₂ group, a C₁₋₅ alkyl, a C₁₋₅ alkoxy, a fused or unfused C₁₋₇ cycloalkyl, a fused or unfused aryl group, possibly substituted by a C₁₋₅ alkyl, a C₁₋₅ alkoxy, a halogen, said C₁₋₅ alkyl group, saturated or unsaturated C₃₋₉ cycloalkyl group or aryl group comprising possibly one or more heteroatoms such as O, N or Si.

or R and R1 together form a saturated or unsaturated C₅₋₂₀ carbocycle such as a cyclopentyl, a cyclohexyl, a cycloheptyl such as a cyclopentadiene, a cyclohexene, a cyclohexadiene, a phenyl, a naphthyl, said carbocycle comprising possibly one or more substituents selected from among a halogen such as chlorine, fluorine or bromine, an —NO₂ group, a C₁₋₅ alkyl, a C₁₋₅ alkoxy, a C₁₋₇ cycloalkyl, a C₅₋₆ aryl, said carbocycle comprising possibly a fusion with a saturated or unsaturated C₅₋₂₀ carbocycle, said C₁₋₅ alkyl group, C₁₋₇ cycloalkyl group, saturated or unsaturated C₅₋₂₀ carbocycle or C₅₋₆ aryl group, are possibly substituted by a halogen such as fluorine, chlorine or bromine, an —NO₂ group, a C₁₋₅ alkyl, a C₁₋₅ alkoxy, a C₁₋₇ cycloalkyl, a C₅₋₆ aryl group, said saturated or unsaturated C₅₋₂₀ carbocycle group, C₁₋₅ alkyl group, C₁₋₇ cycloalkyl group or C₅₋₈ aryl group comprising possibly one or more heteroatoms such as O, N or Si,

n is a whole number comprised between 0 and 2 inclusively,

R2 represents a group selected from among a saturated or unsaturated C₁₋₁₀ alkyl, a saturated or unsaturated C₃₋₉ cycloalkyl, an aryl, a 2-furanyl, a 2-thiophenyl, a 3-thiophenyl or a ferrocenyl, said groups comprising possibly one or more substituents selected from among a halogen such as chlorine, fluorine or bromine, an —NO₂ group, a C₁₋₅ alkyl, a C₁₋₅ alkoxy, a saturated or unsaturated C₁₋₇ cycloalkyl which can be fused or unfused, a polystyryl group, a fused or unfused aryl group which can be optionally substituted by a C₁₋₄ alkyl, a C₁₋₄ alkoxy or a halogen, said groups comprising possibly one or more heteroatoms such as O, N or Si.

A group of preferred derivatives that are useful as ligands according to the invention respond to formula (II) below:

(II)

in which:

R1 and R2 have the same meaning as above and R3, R4, R5, R6 and R7, which can be identical or different, are selected from among a hydrogen atom, a halogen atom such as chlorine, fluorine or bromine, an —NO₂ group, a C₁₋₅ alkyl group, a C₁₋₅ alkoxy group, a fused or unfused C₁₋₇ cycloalkyl group, a fused or unfused aryl group, possibly substituted by a C₁₋₅ alkyl, a C₁₋₅ alkoxy, a halogen, said groups comprising possibly one or more heteroatoms such as O, N or Si.

The invention envisages more specifically the use as ligands of derivatives of formulas (I) or (II) in which:

R1 represents a hydrogen atom, a C₁₋₄ alkyl such as methyl, ethyl, propyl, isopropyl or a phenyl,

R2 represents a group selected from among a 2-furanyl, a 2-thiophenyl, a 3-thiophenyl, a ferrocenyl, an aryl of formula (III) below:

(III)

in which R8, R9, R10, R11 and R12, which can be identical or different, are selected from among a hydrogen atom, a halogen atom such as chlorine, fluorine or bromine, an —NO₂ group, a C₁₋₅ alkyl group, a C₁₋₅ alkoxy group, a fused or unfused C₁₋₇ cycloalkyl group, a fused or unfused aryl group, possibly substituted by a C₁₋₅ alkyl, a C₁₋₅ alkoxy, a halogen, said groups comprising possibly one or more heteroatoms such as O, N or Si.

As an example of the preferred derivatives which are useful as ligands according to the invention, those of formula (IV) below can be cited:

(IV)

in which Ar is a phenyl group carrying one or more substituents such as a halogen, a hydrocarbon group which can be cyclical and/or acyclical, aliphatic and/or aromatic, comprising one or more carbon atoms, and possibly one or more heteroatoms such as O, N and Si, as well as one or more halogens such as F, Cl, Br or I.

The most preferred derivatives which are useful as ligands according to the invention are the biphenyls responding to formula (V) below:

(V)

in which:

R1, R3, R4, R5, R6 and R7 have the same meaning as in formula (I),

R8, R9, R11 and R12 have the same meaning as in formula (III) and

R13, R14, R15, R16 and R17, which can be identical or different, are selected from among a hydrogen atom, a halogen such as chlorine, fluorine or bromine, an —NO₂ group, a C₁₋₅ alkyl, a C₁₋₅ alkoxy, a C₁₋₇ cycloalkyl, a polystyryl group, an aryl group possibly substituted by a C₁₋₄ alkyl, a C₁₋₄ alkoxy or a halogen, said alkyl, alkoxy, cycloalkyl, polystyryl, aryl groups comprising possibly one or more heteroatoms such as O, N or Si.

Among these compounds, the invention envisages most especially the following ligands:

(1S,2R)-N-(4-biphenylmethyl)-norephedrine which will be designated below as derivative “E”,

(1S,2R)-N-(4-ethoxybenzyl)-norephedrine which will be designated below as derivative “F”,

(1S,2R)-N-(4-ethylbenzyl)-norephedrine which will be designated below as derivative “G”,

(1S,2R)-N-(2-chlorobenzyl)-norephedrine which will be designated below as derivative “H”,

(1S,2R)-N-(2-methylbenzyl)-norephedrine which will be designated below as derivative “I”,

(1S,2R)-N-(2,5-dimethylbenzyl)-norephedrine which will be designated below as derivative “J”,

(1S,2R)-N-(1-naphthyl)-norephedrine which will be designated below as derivative “K”,

(1S,2R)-N-(2-thiophenylmethyl)-norephedrine which will be designated below as derivative “L”,

(1S,2R)-N-(1-thiophenylmethyl)-norephedrine which will be designated below as derivative “M”,

(1S,2R)-N-(2-methoxybenzyl)-norephedrine which will be designated below as derivative “N”,

(1S,2R)-N-(1-furanylmethyl)-norephedrine which will be designated below as derivative “O”,

(1S,2R)-N-(4-ferrocenylmethyl)-norephedrine which will be designated below as derivative “P”,

bis-(1S,2R)-N, N′-(1,1′-ferrocenyl)dimethyl)-norephedrine which will be designated below as derivative “Q”

or their optical enantiomers.

The invention also covers the corresponding ligands stemming from the (1R,2S) enantiomer of norephedrine.

In fact, the derivatives of the invention comprise at least two asymmetrical carbons and can thus exist in multiple optically active forms, all of which are covered by the present invention.

The ligands according to the invention can be obtained by a reaction between norephedrine and a substituted derivative of benzaldehyde. For the entirety of the description, norephedrine is understood to mean the (1S,2R) enantiomer of 1-phenyl-2-amino-propan-1-ol. But quite obviously the invention also comprises the ligands stemming from the other enantiomer of norephedrine, i.e. the (1R,2S) enantiomer of 1-phenyl-2-amino-propan-1-ol. The enantiomeric purity of norephedrine is greater than 98%.

Thus, the E, F, G, H, I, J, K, L, M, N, O, P and Q derivatives of the invention were synthesized by a known method, similar to the method employed to synthesize molecules C and D (J. Saavedra, J. Org. Chem. 1985, 50, 2273). Compounds C and D are, respectively:

(1S,2R)-N-benzyl-norephedrine, and

(1S,2R)-N-(4-methoxybenzyl)-norephedrine.

As stated above, the derivatives of formula (I), and preferably the derivatives of formulas (II) and (IV), and most preferably the derivatives of formula (V), and especially preferably the derivatives E, F, G, H, I, J, K, L, M, N, O, P and Q, constitute according to the invention ligands which are effective for hydrogen transfer reduction of carbonyl compounds and enable, according to preferred modes of implementation, production of alcohols with high catalytic activities and, in certain cases, with high enantiomeric purities.

The invention therefore also has its object the use of the derivatives of formulas (I), (II), (IV) and (V), and most especially the derivatives E, F, G. H, I, J, K, L, M, N, O, P and Q, as ligands in a process for the enantioselective reduction of unsaturated compounds carrying functional groups by the hydrogen transfer method. Said unsaturated compounds carrying functional groups are more specifically the carbonyls, imines, iminiums, oximes or compounds comprising a carbon-carbon double bond. The invention pertains most specifically to the use of said derivatives as ligands in a enantioselective reduction process by the hydrogen transfer method of compounds of formula (VI) below:

(VI)

in which,

R18 is selected from among a C₁₋₅ alkyl, an aryl group, a heteroaryl group comprising one or more heteroatoms such as oxygen or nitrogen possibly substituted by a C₁₋₄ alkyl, by a C₁₋₄ alkoxy or by a halogen.

R19 is different from R18 and selected from among an oxyalkyl, an alkoxycarbonyl, an aryl possibly substituted by a C₁₋₄ alkyl, by a C₁₋₄ alkoxy or by a halogen, a heteroaryl, a heteroaryl comprising one or more heteroatoms such as oxygen or nitrogen possibly substituted by a C₁₋₄ alkyl, by a C₁₋₄ alkoxy or by a halogen, and

z represents an oxygen atom, a group of formula —NR20, —NOR₂₀, —N(R₂₀)₂Y or C(R20)₂ in which the R20 groups, which can be identical or different, represent a group selected from among a C₁₋₅ alkyl, an aryl group, a heteroaryl group comprising one or more heteroatoms such as oxygen or nitrogen possibly substituted by a C₁₋₄ alkyl, and Y is a counter anion such as an anionic organic or inorganic molecule, e.g., a halogen, an acetate, a borate, etc.

As examples of derivatives of formula (VI), one can cite the β-ketoesters, the acetylpyridines and the β-alkoxyketones.

The invention thus also pertains to a process for enantioselective reduction of compounds of formula (VI) by a hydrogen transfer method, characterized in that a derivative of formula (I), (II), (IV) or (V), and preferably a derivative E, F or G, is brought to react with a compound of formula (VI) in a basic or neutral medium in the presence of a catalytic quantity of a complex of a transition metal and a secondary alcohol as reducer.

The transition metal is preferably iridium, rhodium or ruthenium, and is advantageously of the type [MCl₂(arene)]₂, in which M represents a transition metal such as rhodium, iridium or ruthenium, and arene means a compound of formula (VII) below:

(VII)

in which R21, R22, R23, R24, R25 and R26, which can be identical or different, are selected from among a hydrogen atom, a halogen, a C₁₋₅ alkyl group, an isoalkyl, a tertioalkyl, an alkoxy, with said alkyl and alkoxy groups comprising one or more heteroatoms such as O, N and Si.

The quantity of substrate of formula (VI) in relation to the catalytic quantity of the complex of a transition metal is from 1 to 50,000, preferably from 10 to 10,000, and most preferably from 100 to 1000.

Optionally, the basic medium in which the process of the invention is performed is implemented advantageously by potassium isopropylate. The hydrogen source in the process of the invention is advantageously a secondary alcohol and preferably isopropanol.

The applicant studied in detail the aforementioned enantioselective reduction process in order to determine which different intermediary products are formed and which can be isolated for the implementation of a variant of said process.

Thus, the bringing into contact:

of the ligand constituted by a derivative of formulas (I), (II), (IV) or (V), preferably a derivative E, F or G. with:

a complex of a transition metal of the type [MCl₂(arene)]₂ and a secondary alcohol,

leads to a first metallic complex of formula (VIII) below:

(VIII)

in which: M, R, R1 and R2 have the same meaning as above, arene indicates a compound of formula (VII) above and R27 represents a halogen such as chlorine or bromine.

The first metallic complex of formula (VIII) leads in a basic medium to the formation of a second metallic complex of formula (IX) below:

(IX)

in which: M, R, R1 and R2 have the same meaning as above, arene indicates a compound of formula (VII) above, and R29 and R28 each represent an electron pair.

The second metallic complex of formula (IX) leads in the presence of a secondary alcohol as reducer to a third metallic complex of formula (X) below:

(X)

in which: M, R, R1 and R2 have the same meaning as above, arene indicates a compound of formula (VII) above.

These metallic compounds can be isolated and used in variants of the process for enantioselective reduction of compounds of formula (VI) by a hydrogen transfer method, which compounds also pertain to the present invention. They can be represented by the general formula (XI) below:

(XI)

in which: M, R, R1 and R2 have the same meaning as above, arene indicates a compound of formula (VII) above, R30 represents a hydrogen atom or an electron pair, R31 represents a hydrogen, a halogen such as chlorine or bromine, or an electron pair.

The invention also has as its object the compounds of formulas (VIII), (IX), (X) and general formula (XI), their preparation and their use in a process for enantioselective reduction of compounds of formula (VI), carriers of functional groups, by a hydrogen transfer method, in the presence or absence of a base.

Thus, the invention has as its object a process for enantioselective reduction of unsaturated compounds which are carriers of functional groups, which compounds are advantageously of formula (VI), by a hydrogen transfer method, characterized in that it comprises the employment of a catalytic quantity of a compound of formula (VIII) in a basic medium and in the presence of a secondary alcohol as reducer.

Thus, the object of the invention is a process for enantioselective reduction of unsaturated compounds that are carriers of functional groups, which compounds are advantageously of formula (VI), by a hydrogen transfer method, characterized in that it comprises the employment of a catalytic quantity of a compound of formula (IX) or (X) in a neutral medium and in the presence of a secondary alcohol as reducer.

More specifically, the invention pertains to the employment of the complex of formula (IX) in a process for the enantioselective reduction of unsaturated compounds that are carriers of functional groups, which compounds are advantageously of formula (VI), by a hydrogen transfer method, comprised of reacting, in the absence of a base, said complex of formula (IX) with a compound of formula (VI) in the presence of a secondary alcohol as reducer. This process presents the advantage of not being implemented in a basic medium.

Other advantages and characteristics of the invention will come to light from the examples below which relate to the preparation of the compounds of formulas (I), (II), (IV) and (V), and especially the compounds E, F and G, and their use in processes for enantioselective reduction of optically active unsaturated derivatives which are carriers of functional groups by the hydrogen transfer method.

### EXAMPLE 1

### Preparation and Characterization of N-substituted Derivatives of Norephedrine

The operating mode below pertains to (1S,2R)-N-(4-biphenylmethyl)-norephedrine (Compound E)

A solution of (1S,2R)-(+)-norephedrine (2.0 g, 13.0 mmol) and 4-biphenylcarboxaldehyde (2.4 g, 13.0 mmol) in 14 ml of ethanol was agitated at room temperature for 15 minutes. Sodium borohydride (0.94 g, 9 mmol) was then added at 0° C. in small portions. The reaction mixture was agitated at 0° C. for 20 minutes; 2.5 ml of water and 5 ml of dichloromethane were then added successively. The resultant solution was filtered on fritted glass and the filtrate was concentrated under vacuum at room temperature. The oily residue obtained was redissolved in 15 ml of ethyl ether and washed with 3×10 ml of dissolved water; the organic phase was then separated out by decantation then dried over MgSO₄ for 2 hours. After filtration and evaporation of the solvent, an oil was recovered and crystallized in heptane at 25° C. Product E was obtained in the form of a white crystalline powder (3.5 g, 85% of yield).

Ligand E exhibits the following characteristics:

White powder. M.p.: 81° C.

¹H NMR (CDCL₃): δ=0.88 (d. 3H. J=6.5. CH₃). 3.03 (dq. 1H. J=3.8. J=6.5 CHNH). 3.93 (s. 2H. CH₂). 4.83 (d. 1H. J=3.8. CHOH). 7.20-7.65 (m. 14H. H_{arom.}).

¹H NMR (C₆D₆): δ=0.71 (d. 3H. J=6.5. CH₃). 2.79 (dq. 1H. J =3.5. CHNH). 3.52 (d. 1H. J=13.4. CHH). 3.59 (d. 1H. J=13.4. CHH). 4.75 (d. 1H. J=3.8. CHOH). 7.0-7.55 (m. 14H. Hₐᵣₒₘ).

¹³C NMR (CDCL₃): δ=14.72 (CH₃). 50.95 (CH₂). 57.83 (CHNH). 73.25 (CHOH). 126.17. 127.09. 127.32. 127.46. 128.14. 128.55. 128.82 (14 CHₐᵣₒₘ). 139.17. 140.17. 140.87. 141.33 (4 C_{q}).

[α]D²⁵=+16.6 (c=1.0; CH₂Cl₂).

MS (CI. NH₃) m/z: 318 [MH+]−MS (EI) m/z (%): 167 (CH₂PhPh. 100%).

Elemental analysis calculated for C₂₂H₂₃NO (317.43): C 83.24. H 7.30. N 4.41; found: C 83.4. H 7.2. N 4.3.

### EXAMPLE 2

### Preparation and Characterization of (1S,2R)-N-(4-ethoxybenzyl)-norephedrine (Compound F)

The procedure specified in example 1 was implemented using 4-ethoxybenzaldehyde (1.95 g, 13.0 mmol) in place of the 4-biphenylcarboxaldehyde. Product F was obtained in the form of a white crystalline powder (2.8 g, 75% of yield).

Ligand F exhibits the following characteristics:

Yield: 75%, slightly yellowish powder. Mp: 59° C.

[α]D²⁵=+15.4 (c=1.0; CH₂Cl₂).

¹H NMR (CDCl₃): δ=0.86 (d. 3H. J=6.7. CH₃). 1.42 (t. 3H. J=7.0. CH₃CH₂). 2.98 (dq. 1H. J=3.8. J=6.7. CHNH). 3.91 (s. 2H. CH₂NH). 4.05 (q. 2H. J=7.0. CH₃CH₂). 4.78 (d. 1H. J=3.8. CHOH). 6.85-6.89 (m. 2H. Hₐᵣₒₘ). 7.20-7.30 (m. 7H. Hₐᵣₒₘ).

¹³C NMR (CDCl₃): δ=14.47. 14.75 (2 CH₃). 50.52 (CH₂NH). 57.53 (CHNH). 63.33 (CH₂CH₃). 73.11 (CHOH). 114.39. 126.01. 126.91. 128.00. 129.13. (9 CHₐᵣₒₘ). 131.91. 141.35. 158.00 (3 C_{q}).

HRMS: m/z calculated for C₁₈H₂₄NO₂ [M+1]⁺: 286.1807; found: 286.1802.

### EXAMPLE 3

### Preparation and Characterization of (1S,2R)-N-(4-ethylbenzyl)-norephedrine (Compound G)

The procedure specified in example 1 was implemented using 4-ethylbenzaldehyde (1.75 g, 13.0 mmol) in place of the 4-biphenylcarboxaldehyde. Product G was obtained in the form of a white crystalline powder (3.08 g).

Ligand G exhibits the following characteristics:

Yield: 88%.

White powder. Mp: 66° C.

¹H NMR (CDCl₃): δ=0.84 (d. 3H. J=6.6. CH₃). 1.24 (t. 3H. J=7.6. CH₃CH₂). 1.55 (s broad. 2 H. OH. NH). 2.65 (q. 2H. J=7.6. CH₃CH₂). 3.00 (dq. 1H. J=3.8. J=6.6. CHNH). 3.85 (s. 2H. CH₂NH). 4.80 (d. 1H. J=3.8. CHOH). 7.15-7.35 (n. 9H. Hₐᵣₒₘ).

¹³C NMR (CDCl₃: δ=14.63. 15.58 (2 CH₃). 28.48 (CH₂CH₃). 50.97 (CH₂NH). 57.69 (CHNH). 72.99 (CHOH). 126.06. 126.98. 128.02 (9 CHₐᵣₒₘ). 137.25. 141.26. 143.16. (3 C_{q}).

[a]D²⁵=+18.6 (c=1.0; CH₃Cl₂).

HRMS m/z calculated for C₁₈H₂₄NO [M+1]⁺: 270.1858; found: 270.1852.

### EXAMPLE 3′

### (1S,2R)-N-(cyclohexylmethyl)-norephedrine

The procedure specified in example 1 was implemented using cyclohexanecarboxaldehyde in place of the 4-biphenylcarboxaldehyde. The product was obtained in the form of colorless needles.

This compound exhibits the following characteristics:

Yield: 80%. Colorless needles. Mp: 90° C.

¹H NMR (CDCl₃): δ=0.79 (d. 3H. J=6.5. CH₃). 0.83-0.99 (m. 2H. Cy). 1.10-1.32 (m. 3H. Cy). 1.34-1.48 (m. 1H. Cy). 1.63-1.84 (m. 5H. Cy). 2.47 (dd. 1H. J=6.9. J=11.5. CHHNH). 2.57 (dd. 1H. J=6.3. J=11.5. CHHNH). 2.90 (dq. 1H. J=3.9. J=6.5. CH₃CH). 4.72 (d. 1H. J=3.9. CHOH). 7.20-7.35 (m. 5H. Hₐᵣₒₘ).

¹³C NMR (CDCl₃): δ=14.79 (CH₃). 25.99. 26.62. 31.45. (5 CH₂. Cy). 38.31 (CH. Cy). 53.96 (CH₂NH). 58.43 (CHNH). 72.72 (CHOH). 125.99. 126.84. 127.95 (5 CHₐᵣₒₘ). 141.43 (1 C_{q}).

[β]D²⁵=+5.0 (c=1.0; CH₂Cl₂).

Elemental analysis calculated for C₁₆H₂₅NO (247.38): C 77.68. H 10.18. N 5.66; found: C. H. N.

### EXAMPLES 4 to 20

### Use of the Ligands of the Invention in the Reduction by Hydrogen Transfer of tert-butyl acetoacetate

The results obtained in the presence of ligands A, B, C and D are presented for comparison. Identical operating conditions as described below for example 6 were employed in all cases.

The complex [RuCl₂(η⁶-benzene)₂ (5.0 mg, 0.01 mmol) and (1S,2R)-N-benzyl-norephedrine (9.7 g, 0.04 mmol) were introduced into a Schlenk tube that had been purged by three vacuum/nitrogen cycles. The solids were dissolved in 5 ml of distilled isopropanol. The mixture was degassed by freezing under vacuum before being placed under a nitrogen atmosphere and then brought to 80° C. for 20 minutes. The solution took on an orange coloration. After rapid cooling, there were introduced successively under nitrogen using a cannula: a previously degassed solution of tert-butyl acetoacetate (316 mg, 2.0 mmol, 100 eq./Ru) in 14 ml of isopropanol, then 1 ml of a degassed solution at 0.12 mol/l of potassium isopropylate. All of this was then agitated magnetically under nitrogen at 23° C. The progress of the reaction was monitored by gas-phase chromatography using an achiral column (Chirasil DEX-C).

The results of the reduction reactions are presented in Table 1 below.

TABLE 1

| Exam- | [RuCl₂(arene)]₂ | | Duration | Conversion | t_{1/2} | Enantiomeric | |
|---|---|---|---|---|---|---|---|
| ple | (arene) | Ligand | (h) | (mol-%) | (min) | excess (%) | Configuration |
| 4 | benzene | A | 1 | 98 | 16 | 44 | S−(+) |
| 5 | benzene | B | 17 | 21 | n.d | <5 | S−(+) |
| 6 | benzene | C | 5 | 100 | 135 | 68 | S−(+) |
| 7 | benzene | D | 4 | 100 | 60 | 66 | S−(+) |
| 8 | benzene | E | 2.5 | 100 | 65 | 67 | S−(+) |
| 9 | benzene | F | 3 | 100 | 65 | 61 | S−(+) |
| 10 | benzene | G | 8 | 100 | 130 | 60 | S−(+) |
| 11 | benzene | H | 13 | 38 | n.d | 63 | S−(+) |
| 12 | benzene | I | 19 | 43 | n.d | 62 | S−(+) |
| 13 | benzene | J | 31 | 100 | 540 | 59 | S−(+) |
| 14 | benzene | K | 6 | 4 | n.d | 28 | S−(+) |
| 15 | benzene | L | 5 | 100 | 120 | 61 | S−(+) |
| 16 | benzene | M | 6 | 100 | 110 | 59 | S−(+) |
| 17 | benzene | N | 16 | 70 | 600 | 64 | S−(+) |
| 18 | benzene | O | 4 | 100 | 60 | 56 | S−(+) |
| 19 | benzene | P | 4 | 100 | 90 | 60 | S−(+) |
| 20 | benzene | Q | 5.5 | 100 | 110 | 57 | S−(+) |

### EXAMPLES 21 to 23

### Use of the Ligands of the Invention in the Reduction by Hydrogen Transfer of ethyl acetoacetate

The method used for examples 4 to 20 was employed, working at 50° C., using acetoacetate (263 mg, 2.0 mmol) in place of tert-butyl acetoacetate.

The results of the reduction reactions are presented in Table 2 below.

TABLE 2

| Exam- | [RuCl₂(arene)]₂ | | Duration | Conversion | t_{1/2} | Enantiomeric | |
|---|---|---|---|---|---|---|---|
| ple | (arene) | Ligand | (h) | (mol-%) | (min) | excess (%) | Configuration |
| 21 | benzene | A | 0.5 | 100 | 10 | 15 | S−(+) |
| 22 | benzene | B | 3 | 100 | 70 | 15 | S−(+) |
| 23 | benzene | C | 1 | 100 | 10 | 56 | S−(+) |
| 24 | benzene | D | 0.5 | 10 | 10 | 56 | S−(+) |
| 25 | benzene | E | — | — | — | 58 | — |
| 26 | benzene | F | — | — | — | 54 | — |
| 27 | benzene | G | 0.7 | — | 30 | 55 | — |

### EXAMPLES 28 to 36

### Use of the Ligands of the Invention in the Reduction by Hydrogen Transfer of 2-acetylpyridine

The method used for examples 4 to 20 was employed using 2-acetylpyridine 242 mg, 2.0 mmol) in place of tert-butyl acetoacetate.

The results of the reduction reactions are presented in Table 3 below.

TABLE 3

| Exam- | [RuCl₂(arene)]₂ | | Duration | Conversion | t_{1/2} | Enantiomeric | |
|---|---|---|---|---|---|---|---|
| ple | (arene) | Ligand | (h) | (mol-%) | (min) | excess (%) | Configuration |
| 28 | p-cymene | A | 0.5 | 100 | 8 | 83 | R−(−) |
| 29 | p-cymene | B | 17 | 43 | n.d. | 84 | R−(−) |
| 30 | p-cymene | C | 16 | 100 | 420 | 89 | R−(−) |
| 31 | p-cymene | E | 6 | 100 | 120 | 88 | R−(−) |
| 32 | benzene | A | 0.5 | 100 | 7 | 68 | R−(−) |
| 33 | benzene | C | 2 | 100 | 15 | 79 | R−(−) |
| 34 | benzene | E | 0.5 | 97 | 10 | 78 | R−(−) |
| 35 | tert-butyl | E | 15 | 100 | 180 | 83 | R−(−) |
| | benzene | | | | | | |
| 36 | anisole | E | 1 | 98 | 10 | 78 | R−(−) |
| 37 | p-cymene | F | 7 | 98 | 120 | 86 | S−(−) |
| 38 | benzene | F | 0.5 | — | 10 | 77 | — |

### EXAMPLES 39 to 42

### Use of the Ligands of the Invention in the Reduction by Hydrogen Transfer of 2-methoxyacetone

The method used for examples 4 to 20 was employed using methoxyacetone 178 mg, 2.0 mmol) in place of tert-butyl acetoacetate.

The results of the reduction reactions are presented in Table 4 below.

TABLE 4

| Exam- | [RuCl₂(arene)]₂ | | Duration | Conversion | t_{1/2} | Enantiomeric | |
|---|---|---|---|---|---|---|---|
| ple | (arene) | Ligand | (h) | (mol-%) | (min) | excess (%) | Configuration |
| 39 | benzene | A | 0.33 | 100 | 5 | 51 | n.d. |
| 40 | benzene | B | 16 | 39 | n.d. | n.d. | n.d. |
| 41 | benzene | C | 0.33 | 100 | 5 | 66 | n.d. |
| 42 | benzene | E | 0.33 | 100 | 5 | 66 | S−(−) |
| 43 | benzene | F | — | 100 | — | 62 | — |
| 44 | — | G | — | — | — | 63 | — |
| 45 | anisole | E | — | — | — | 61 | — |
| 46 | tetraline | E | — | — | — | 52 | — |

### EXAMPLES 47 to 51

Examples 47 to 51 below describe the preparation and characterization of precursor complexes VIIIa-c and catalytically active complexes IXa and Xa from the complex [RuCl₂(η⁶-p-cymene)]₂ and β-amino alcohol ligands.

IIa-c IXa Xa

a: R=

b: R=Me

c: R=

### EXAMPLE 47

### [RuCl{η⁶-p-cymene}{η²-(1S,2R)—N-(4-biphenylmethyl)-norephedrine)} (VIIIa)

A solution of [RuC₂(η⁶-p-cymene)₂ (612.5 mg, 1.0 mnmol), (1S,2R)—N-(4-biphenylmethyl)-norephedrine (derivative “E”, 634 mg, 2.0 mmol) and triethylamine (0.56 ml, 4.0 mmol) in 2-propanol (20 ml) was heated at 80° C. for 2 h. The resultant orange solution was concentrated to dryness, the residue was washed with water (2 times 4 ml), then dried under vacuum so as to yield compound VIIa in the form of a brown powder. The yield obtained was 66%. The same complex was prepared by agitating a solution of the compound according to IXa in chloroform for 30 minutes then drying under vacuum. The yield then was 100%. Compound VIIIa was characterized by its infrared spectrum, NMR, mass spectrometry and X-ray diffraction as follows: IR (KBr): v [cm⁻¹]: 3195 (H—N). —¹H NMR (C₆D₆): δ=0.56 (d, 3H, J=6.3, CH₃CHN), 1.17, 1.20 (each d, 3H, J=7.1, CH(CH₃)₂), 2.02 (s, 3H, CH₃ of the p-cymene), 2.39 (m, 1H, CHNH), 2.82 (m, 1H, CH(CH₃)₂), 3.82 (dd, 1H, J=11.0, J=13.6, CHHNH), 4.34 (dd, 1H, J=4.2, J=13.6, CHHNH), 4.54, 4.64 (each d, 1H, J=5.5, Hₐᵣₒₘ of the p-cymene), 4.87 (broad d, 1H, J=10.5, NH), 5.06 (d, 1H, J=2.6, PhCH), 5.12, 5.20 (each d, 1H, J=5.8, Hₐᵣₒₘ of the p-cymene), 7.0-7.65 (m, 14H, Hₐᵣₒₘ). —¹³C NMR (C₆D₆): δ=8.66 (CH₃CHN), 17.02 (CH₃ of the p-cymene), 21.64, 23/74 (CH(CH₃)₂), 31.11 (CH(CH₃)₂), 56.18 (CH₂NH), 60.18 (CHNH), 77.25, 77.95, 80.24 (3 CHₐᵣₒₘ of the p-cymene), 81.59 (PhCH), 82.34 (CHₐᵣₒₘ of the p-cymene), 94.57, 101.03 (2 C_{q} of the p-cymene), 126.08, 127.34, 127.45, 129.15 (14 CHₐᵣₒₘ), 137.87, 140.19, 141.30, 142.43 (4 C_{q}). —¹H NMR (C₆D₅CD₃): δ=0.58 (d, 3H, J=6.3, CH₃CHN), 1.21, 1.23 (each d, 3H, J=7.2, CH(CH₃)₂), 2.08 (s, 3H, CH₃ of the p-cymene), 2.37a (m, 1H, CHNH), 2.86 (m, 1H, CH(CH₃)₂), 3.87 (dd, 1H, J=11.3, J=13.6, CHHNH), 4.38 (dd, 1H, J=3.8, J=13.6, CHHNH), 4.38 (dd, 1H, J=3.8, J=13.6, CHHNH), 4.60, 4.68 (each d, 1H, J=5.31 Hₐᵣₒₘ of the p-cymene), 4.89 (broad d, 1H, J=10.6, NH), 5.01 (d, 1H, J=2.9, PhCH), 5.25, 5.33 (each d, 1H, J=5.6, Hₐᵣₒₘ, of the p-cymene), 6.95-7.5 (m, 14H, Hₐᵣₒₘ). —¹H NMR (CDCl₃): δ=0.65 (d, 3H, J=6.2, CH₃CHN), 1.32, 1.35 (each d, 3H, J=7.2, CH(CH₃)₂), 2.29 (m, 4H, CHNH+CH₃ of the p-cymene), 2.94 (m, 1H, CH(CH₃)₂), 4.30 (dd, 1H, J=11.8, J=13.5, CHHNH), 4.55 (m, 2H, PhCH+NH), 4.70 (dd, 1H, J=3.2 and 13.5, CHHNH), 5.14, 5.22 (each d, 1H, J=5.3, Hₐᵣₒₘ of the p-cymene), 5.30, 5.41 (each d, 1H, J=5.8, Hₐᵣₒₘ of the p-cymene), 7.0-7.7 (m, 14H, Hₐᵣₒₘ). —¹³C NMR (CDCl₃): δ=8.18 (CH₃CHN), 17.00 (CH₃ of the p-cymene), 21.50, 23.75 (CH(CH₃)₂), 30.73 (CH(CH₃)₂), 56.17 (CH₂NH), 59.72 (CHNH), 76.74, 78.34, 79.57 (3 CHₐᵣₒₘ of the p-cymene), 81.09 (PhCH), 82.55 (CHₐᵣₒₘ of the p-cymene), 95.34, 101.26, (2 C_{q} of the p-cymene), 125.93, 126.92, 127.01, 127.22, 127.60, 127.73, 128.68, 128.84, (CHₐᵣₒₘ), 137.87, 140.19, 141.30, 142.43 (4 C_{q}). —ESI-MS: m/z (%): 588.1 ([MH]⁺, the relative masses and intensities observed are in perfect agreement with the profile calculated for the anticipated protonated molecule C₃₂H₃₇NOClRu).

### EXAMPLE 48

### [RuCl{η⁶-p-cymene}{η²-(1S,2R)-ephedrine)] (VIIIb)

This compound was synthesized in a manner similar to the chloroform method employed for VIIIa, from ephedrine (compound “A”) and [RuCl₂,η⁶-p-cymene)]₂; compound VIIIb was obtained in the form of a brown powder with a yield of 100%. —¹H NMR (C₆D₆): δ=0.31 (d, 3H, J=6.6, CH₃CHN), 1.14, 1.22 (each d, 3H, J=6.9, CH(CH₃)₂), 2.03 (s, 3H, CH₃ of the p-cymene), 2.09 (m, 1H, CHNH), 2.17 (d, 3H, J=6.4, CH₃NH), 2.88 (m, 1H, CH(CH₃)₂), 4.03 (broad m, 1H, NH), 4.27, 4.48 (each d, 1H, J=5.4, Hₐᵣₒₘ of the p-cymene), 5.03 (d, 1H, J=3.1, PhCH), 5.27, 5.34 (each d, 1H, J=6.0, Hₐᵣₒₘ of the p-cymene), 7.17 (d, 1H, J=7.5, Hₐᵣₒₘ), 7.33 (t, 2H, J=7.5, Hₕₐᵣₒₘ), 7.68 (d, 2H, J=7.5, Hₐᵣₒₘ). —₁₃C NMR (C₆D₆): δ=8.15 (CH₃CHN), 16.77 (CH₃ of the p-cymene), 21.26, 23.97 (CH(CH₃)₂), 30.99 (CH(CH₃)₂), 39.70 (CH₃NH), 64.36a (CHNH), 76.50, 77.45, 79.49 (3 CHₐᵣₒₘ of the p-cymene), 81.21 (PhCH), 82.88 (CHₐᵣₒₘ of the p-cymene), 94.74, 100.14 (2 C_{q} of the p-cymene), 126.11, 127.43, 127.76 (5 CHₐᵣₒₘ), 144.55 (C_{q}).

### EXAMPLE 49

### [RuCl{η⁶-p-cymene}{η²-(1S,2R)—N-benzyl-norephedrine}] (VIIIc)

This compound was synthesized in a manner like that of the chloroform method employed for VIIIa, from (1S,2R)-N-benzyl-norephedrine and [RuCl₂(η⁶-p-cymene)]₂; compound VIIIc was obtained in the form of a brown powder with a yield of 75%. —¹H NMR (CDCl₃): δ=0.62(d, 3H, J=6.4, CH₃CHN), 1.33, 1.36a (each d, 3H, J=7.4, CH(CH₃)₂), 2.24 (m, 1H, CHNH), 2.30 (s, 3H, CH₃ of the p-cymene), 2.96 (m, 1H, CH(CHs)₂), 4.24 (dd, 1H, J=11.3, J=13.4, CHHNH), 4.51 (m, 1H, NH), 4.56 (d, 1H, J=3.1, PhCH), 4.68 (dd, 1H, J=3.7, J=13.4, CHHNH), 5.13, 5.23 (each d, 1H, J=5.6, Hₐᵣₒₘ of the p-cymene), 5.30, 5.45 (each d, 1H, J=6.2, Hₐᵣₒₘ of the p-cymene), 7.05-7.45 (m, 10 H, Hₐᵣₒₘ). —¹³C NMR (CDCl₃): δ=8.00 (CH₃CHN), 16.83 (CH₃ of the p-cymene), 21.36, 23.61 (CH(CH₃)₂), 30.59 (CH(CH₃)₂), 56.22 (CH₂NH), 59.39 (CHNH), 77.20, 78.26, 79.38 (3 CHₐᵣₒₘ of the p-cymene), 80.92 (PhCH), 82.37a (CHₐᵣₒₘ of the p-cymene), 95.13, 101.00 (2 C_{q} of the p-cymene), 125.73, 126.76, 127.04, 128.10, 128.18, 128.92 (10 CHₐᵣₒₘ), 135.82,142.38 (2 C_{q}).

### EXAMPLE 50

### [Ru{η⁶-p-cymene}{η²-(1S,2R)—N-(4-biphenylmethyl)-norephedrine}] (IXa)

A mixture of [RuCl₂(η⁶-p-cymene)]₂ (612 mg, 1.0 mmol), (1S,2R)-N-(4-biphenylmethyl)-norephedrine (634 mg, 2.0 mmol) and KOH (800 mg, 14.3 mmol) in CH₂Cl₂ (14 ml) was heated at 40° C. for 20 min. Water (14 ml) was added to the orange solution which was then agitated at 40° C. for an additional 20 min. The dark brown organic phase was washed with water (14 ml), dried over CaH₂, filtered and concentrated to dryness to yield compound VIIIa in the form of a bright violet powder. The yield was 75%. The same compound was obtained by treating complex VIIIa with an equivalent of KOH in CH₂Cl₂ at 40° C. for 20 minutes and then proceeding in the same manner as described for the preceding operating mode; the yield was then 70%. —1H NMR (C₆D₅CD₃, −20° C.): δ=0.70 (d, 3H, J=6.0, CH₃CHN), 1.26, 1.30 (each d, 3H, J=6.8, CH(CH₃)₂), 1.92 (s, 3H, CH₃ of the p-cymene), 2.44 (m, 1H, CH(CH₃)₂), 2.67 (m, 1H, CHNH), 4.14 (d, 1H, J=15.4, CHHNH), 4.49, 4.83 (each d, 1H, J=5.2, Hₐᵣₒₘ of the p-cymene), 4.9-5.1 (m, 4H, PhCH+CHHNH+2Hₐᵣₒₘ of the p-cymene), 6.9-7.7 (m, 14H, Hₐᵣₒₘ). —¹H NMR (C₆D₆, +20° C.): δ=0.70 (d, 3H, J=6.2, CH₃CHN), 1.23, 1.25 (each d, 3H, J=6.9, CH(CH₃)₂), 1.82 (s, 3H, CH₃ of the p-cymene), 2.43 (m, 1H, CH(CH₃)₂), 2.70 (m, 1H, CHNH), 4.17 (d, 1H, J=14.4, CHHNH), 4.54, 4.89 (each d, 1H, J=5.4, Hₐᵣₒₘ of the p-cymene), 4.9-5.1 (m, 4H, PhCH+CHHNH+2Hₐᵣₒₘ of the p-cymene), 7.0-7.7 (m, 14H, Hₐᵣₒₘ). —¹³C NMR (C₆D₅CD₃, −20° C.): δ=9.69 (CH₃CHN), 16.08 (CH₃ of the p-cymene), 23.94, 24.15 (CH(CH₃)₂), 32.53 (CH(CH₃)₂), 68.56 (CH₂NH), 73.09, 76.36, 77.60, 78.99 (4 CHₐᵣₒₘ), 79.67 (1 CHₐᵣₒₘ+1 C_{q} of the p-cymene), 96.36 (C_{q} of the p-cymene), 126.07, 127.38, 127.60 (CHₐᵣₒₘ), 140.18, 140.85, 141.58, 146.05 (4 C_{q}). —ESI-MS: m/z (%): 552.1 ([MH]⁺, the relative masses and intensities observed are in perfect agreement with the profile calculated for the anticipated protonated molecule C₃₂H₇NOClRu).

### EXAMPLE 51

### [RuH{η⁶-p-cymene}{η²-(1S,2R)—N-(4-biphenylmethyl)-norephedrine}] (Xa)

The violet complex VIIIa (220 mg, 0.4 mmol) was agitated in 2-propanol (7 ml) for 5 min at room temperature. The resultant red solution was immediately concentrated to dryness at −10° C to yield compound Xa in the form of a reddish brown powder. The yield was 100%. —¹H NMR (C₆D₅CD₃, −20° C.): δ=−5.20 (s, 1H, RuH), 0.87 (d, 3H, J=6.2, CH₃CHN), 1.19, 1.35 (each d, 3H, J=6.8, CH(CH₃)₂), 2.17 (s, 3H, CH₃ of the p-cymene), 2.27 (m, 1H, CH(CH₃)₂), 2.33 (m, 1H, CHNH), 3.58 (dd, 1H, J=10.5, J=14.3, CHHNH), 3.71 (dd, 1H, J=3.8, J=14.3, CHHNH), 3.90 (d, 1H, J=5.3 Hz, Hₐᵣₒₘ of the p-cymene), 4.36a (m, 1H, PhCH), 4.72 (d, 1H, J=5.6 Hz, Hₐᵣₒₘ of the p-cymene), 4.81 (m, 1H, NH), 5.17 (d, 1H, J=5.3 Hz, Hₐᵣₒₘ of the p-cymene), 5.46 (d, 1H, J=5.6 Hz, Hₐᵣₒₘ of the p-cymene), 6.8-7.6 (m, 14H, Hₐᵣₒₘ). —¹H NMR (C₆D₆, +20° C.): δ=−5.11 (s, 1H, RuH), 0.90 (d, 3H, J=6.2, CH₃CHN), 1.20, 1.33 (each d, 3H, J=6.9, CH(CH₃)₂), 2.11 (s, 3H, CH₃ of the p-cymene), 2.35 (m, 2H, CHNH+CH(CH₃)₂), 3.76 (m, 2H, CH₂NH), 4.04 (d, 1H, J=5.3, Hₐᵣₒₘ of the p-cymene), 4.43 (m, 1H, PhCH), 4.54 (d, 1H, J=5.31 Hₐᵣₒₘ of the p-cymene), 4.69 (m, 1H, NH), 5.16, 5.38 (each d, 1H, J=5.3, Hₐᵣₒₘ of the p-cymene), 6.8-7.7 (m, 14H, Hₐᵣₒₘ). —¹³C NMR (C₆D₅CD₃, −40° C.): δ=8.16 (CH₃CHN), 19.08 (CH₃ of the p-cymene), 24.69 (CH(CH₃)₂), 33.41 (CH(CH₃)₂), 58.48 (CH₂NH), 60.87 (CHNH), 75.16, 76.19, 78.55 (3 CHₐᵣₒₘ of the p-cymene), 85.18 (PhCH), 88.68 (CHₐᵣₒₘ of the p-cymene), 97.61, 104.39 (2 C_{q} of the p-cymene), 127.41, 128.22, 128.33 (C_{Harom}), 136.88, 141.70, 141.87, 145.23 (4 C_{q}).

Examples 52 to 56 below illustrate the application of the precursor complex VIIIa in enantioselective hydrogen transfer on prochiral ketones.

### Example 52

A solution of 2-acetylpyridine (141 mg, 1.0 mmol) in isopropanol (10 ml) was added to a Schlenk tube containing VIIIa (6.0 mg, 0.01 mmol of Ru). To the resultant orange solution was added 3 equivalents of potassium isopropylate (0.03 mmol, i.e., 250 μl of a 0.12 M solution in iPrOH); the solution quickly turned violet and was agitated magnetically at room temperature; analyzing the solution by gas chromatography showed the formation of (2-pyridyl)-2-ethanol at the level of 55% after 3 h; the conversion was total after 8 h. The alcohol was obtained with a chemoselectivity of 100% and an enantiomeric excess of 88% (S).

### EXAMPLE 53

The same procedure as in example 52 was followed but without the addition of potassium isopropylate. After 4 h of agitation at room temperature, the conversion of the 2-acetylpyridine into alcohol was less than 2%.

### EXAMPLE 54

The same procedure as in example 52 was followed using 12 mg of complex VIIIa (0.02 mmol of Ru), 240 mg (2.0 mmol) of acetophenone in place of the 2-acetylpyridine, and 1.0 equivalent of iPrOK (vs Ru) (0.02 mmol, i.e., 167 μl of a 0.12 M solution in iPrOH). After 10 min, the conversion of the acetophenone into phenyl-2-ethanol had reached 51%; the conversion reached 94% after 1 h. The enantiomeric excess of the alcohol formed was 91% (S).

### EXAMPLE 55

The same procedure as in example 54 was followed but without adding potassium isopropylate. After 4 h of agitation at room temperature, the conversion of the acetophenone into phenyl-2-ethanol was less than 2%.

### EXAMPLE 56

The same procedure as in example 54 was followed using 316 mg (2.0 mmol) tert-butyl acetoacetate in place of the acetophenone. After 4 h, the conversion into tert-butyl 3-hydroxybutyrate was 47%; the conversion was total after 16 h. the enantiomeric excess of the alcohol formed was 30% (S).

Examples 57 and 58 below illustrate the application of the catalytic complex IXa for enantioselective hydrogen transfer on prochiral ketones in the absence of base:

### EXAMPLE 57

A solution of acetophenone (240 mg, 2.0 mmol) in isopropanol (20 ml) was added to a Schlenk tube containing IXa (14.0 mg; 0.025 mmol of Ru). The violet solution was agitated magnetically at room temperature and analyzed by gas chromatography. The formation of phenyl-2-ethanol at the level of 45% was seen after 10 min and at the level of 93% after 1 h. The alcohol was obtained with a chemoselectivity of 100% and an enantiomeric excess of 91% (S).

### EXAMPLE 58

The procedure of example 57 was followed using 316 mg (2.0 mmol) of tert-butyl acetoacetate instead of acetophenone. After 4 h, the conversion into tert-butyl hydroxybutyrate was 51%; the conversion was total after 14 h; the alcohol was obtained with an enantiomeric excess of 30% (S).

Example 59 below illustrates the application of the catalytic complex IXa in the enantioselective hydrogen transfer of acetophenone in the absence of base.

### EXAMPLE 59

The procedure of example 58 was followed using complex Xa in place of complex IXa. The same results were observed.

### EXAMPLE 60

### X-ray structures of the Ligand E.HCl of Example 1 and the Complex VIIIa.MeOH of Example 47

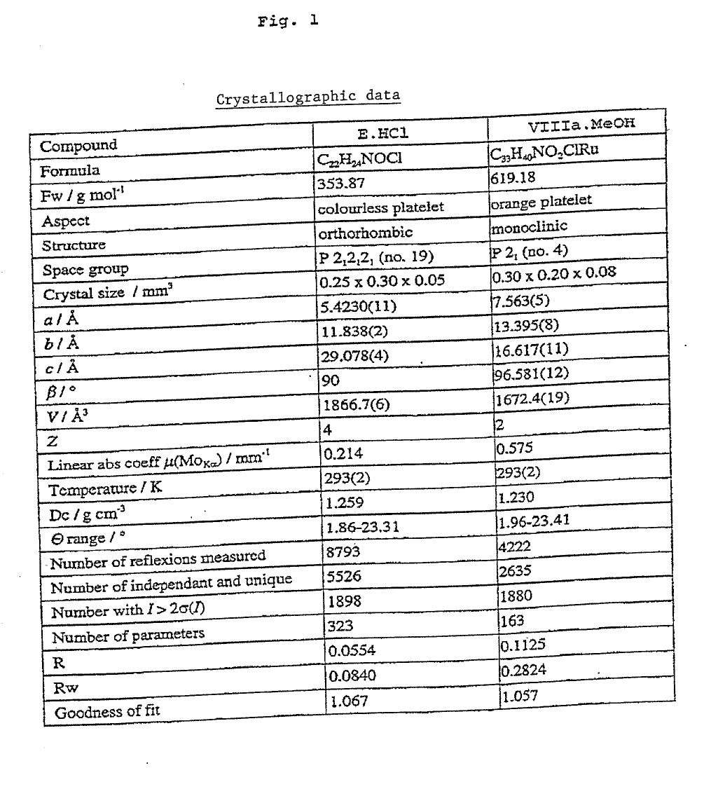

The X-ray diffraction spectra were determined on a BRUKER SMART diffractometer (λ Mo Kα=0.71069 Å, graphite monochromator, T=294 K). The structures were obtained by the direct method (SHELX-97). For ligand E.HCl (example 1), the hydrogen atoms were obtained on the Fourier difference map and their positions were refined in an isotropic manner. For the complex VIIIa.MeOH (example 47), the Ru and Cl atoms were refined in an anisotropic manner whereas the N. O and C atoms were refined in an isotropic manner. Because of the slow decomposition of this compound, the data were recorded using a rapid procedure: 240 intervals of 1.5° of breadth and 20 s of exposure time. The crystallographic data for the two compounds are presented in the table of attached FIG. 1.

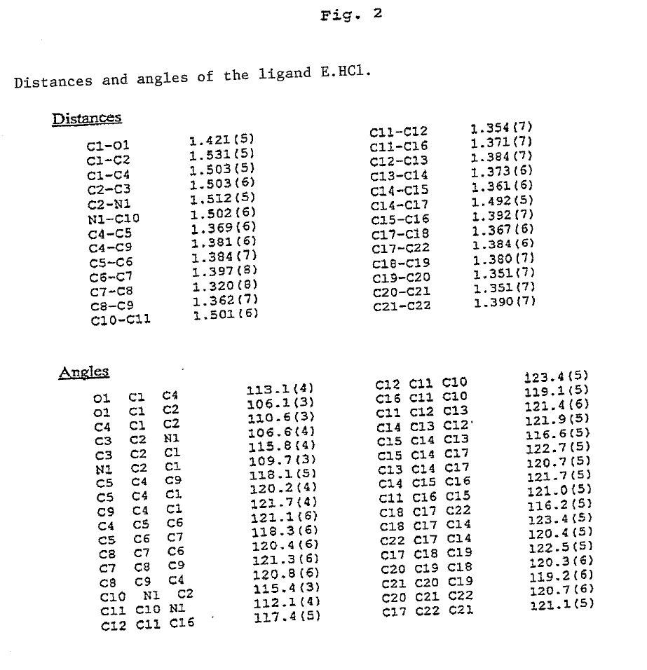

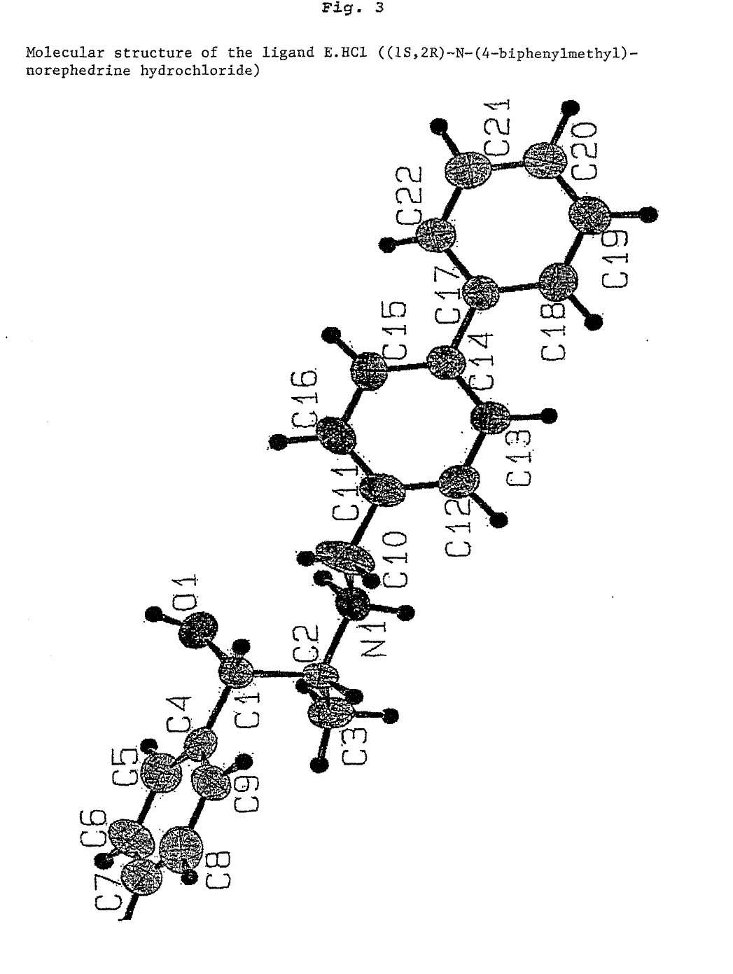

The distances and angles for the ligand E.HCl are presented in attached FIG. 2 and its molecular structure is represented in attached FIG. 3.

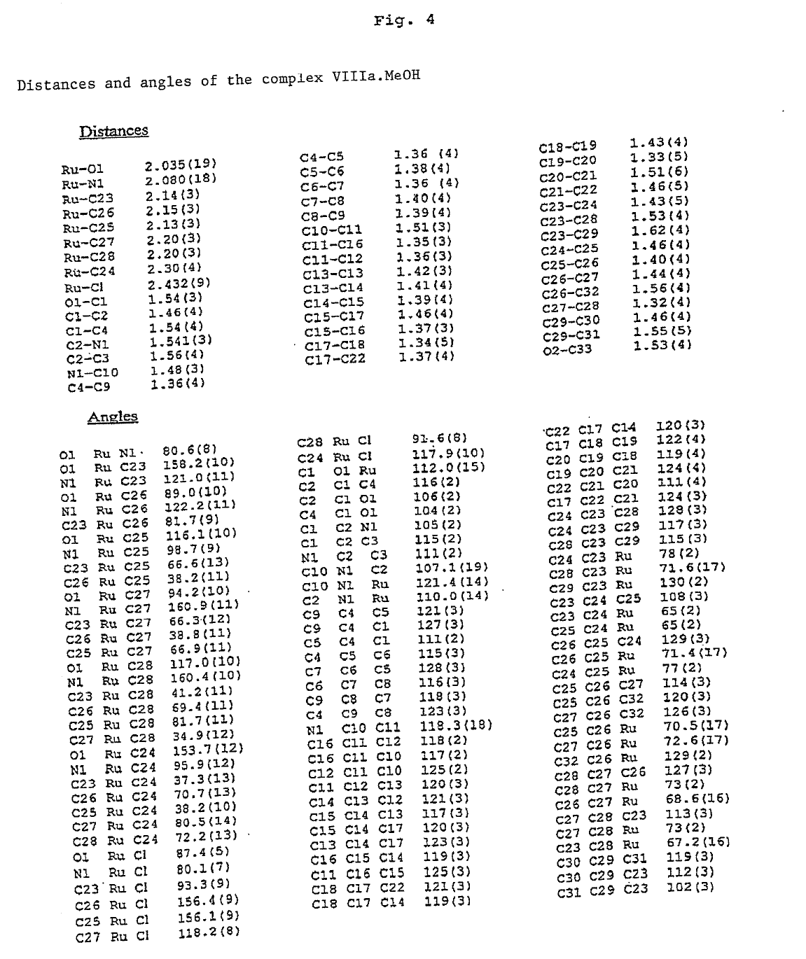

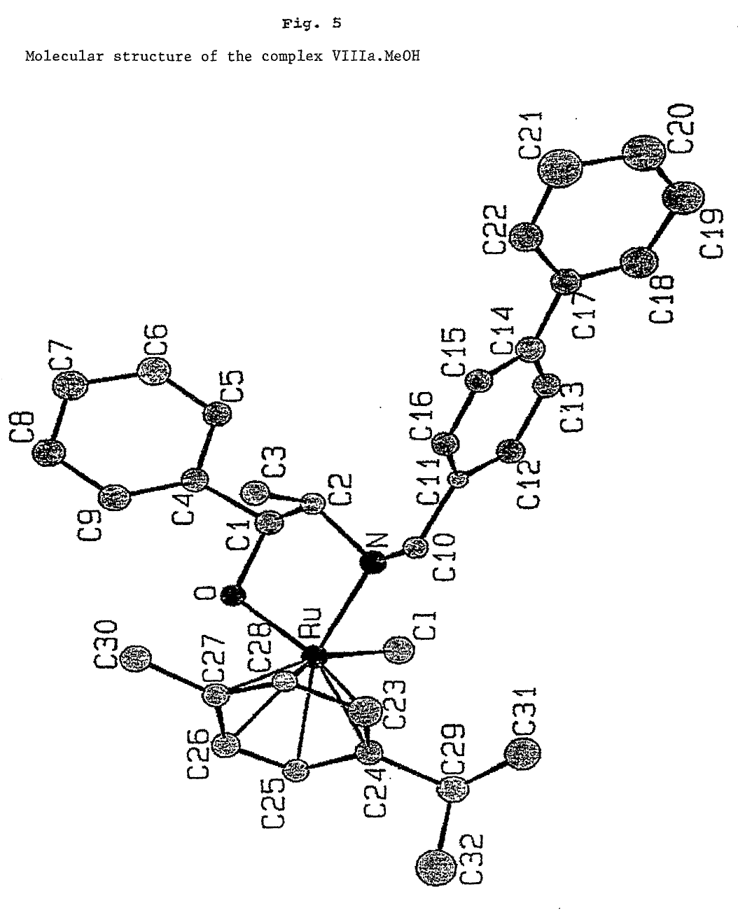

The distances and angles for the complex VIIIa.MeOH are presented in attached FIG. 4 and its molecular structure is represented in attached FIG. 5.

1. Use of an optically active N-substituted chiral derivative of norephedrine of formula (I) below:
(I)
in which:
R represents a C₁₋₁₀ alkyl group, a saturated or unsaturated C₃₋₉ cycloalkyl group, an aryl group, said groups comprising possibly one or more substituents chosen from among a halogen atom such as chlorine, fluorine or bromine, an —NO₂ group, a C₁₋₅ alkyl, a C₁₋₅ alkoxy, a fused or unfused C₁₋₇ cycloalkyl, a fused or unfused aryl group, possibly substituted by a C₁₋₅ alkyl, a C₁₋₅ alkoxy, a halogen, said C₁₋₁₀ alkyl group, saturated or unsaturated C₃₋₉ cycloalkyl group or aryl group comprising possibly one or more heteroatoms such as O, N or Si.
R1 represents a hydrogen atom, a C₁₋₁₀ alkyl group such as methyl, ethyl, propyl or isopropyl, an aryl group such as a phenyl, a saturated or unsaturated C₃₋₉ cycloalkyl group, said groups comprising possibly one or more substituents selected from among a halogen atom such as chlorine, fluorine or bromine, an —NO₂ group, a C₁₋₅ alkyl, a C₁₋₅ alkoxy, a fused or unfused C₁₋₇ cycloalkyl, a fused or unfused aryl group, possibly substituted by a C₁₋₅ alkyl, a C₁₋₅ alkoxy, a halogen, said C₁₋₅ alkyl group, saturated or unsaturated C₃₋₉ cycloalkyl group or aryl group comprising possibly one or more heteroatoms such as O, N or Si.
or R and R1 together form a saturated or unsaturated C₅₋₂₀ carbocycle such as a cyclopentyl, a cyclohexyl, a cycloheptyl such as a cyclopentadiene, a cyclohexene, a cyclohexadiene, a phenyl, a naphthyl, said carbocycle comprising possibly one or more substituents selected from among a halogen such as chlorine, fluorine or bromine, an —NO₂ group, a C₁₋₅ alkyl, a C₁₋₃ alkoxy, a C₁₋₇ cycloalkyl, a C₅₋₆ aryl, said carbocycle comprising possibly a fusion with a saturated or unsaturated C₅₋₂₀ carbocycle, said C₁₋₅ alkyl group, C₁₋₇ cycloalkyl group, saturated or unsaturated C₅₋₂₀ carbocycle or C₅₋₆ aryl group, are possibly substituted by a halogen such as fluorine, chlorine or bromine, an —NO₂ group, a C₁₋₅ alkyl, a C₁₋₅ alkoxy, a C₁₋₇ cycloalkyl, a C₅₋₆ aryl group, said saturated or unsaturated C₅₋₂₀ carbocycle group, C₁₋₅ alkyl group, C₁₋₇ cycloalkyl group or C₅₋₈ aryl group comprising possibly one or more heteroatoms such as O, N or Si,
n is a whole number comprised between 0 and 2 inclusively,
R2 represents a group selected from among a saturated or unsaturated C₁₋₁₀ alkyl, a saturated or unsaturated C₃₋₉ cycloalkyl, an aryl, a 2-furanyl, a 2-thiophenyl, a 3-thiophenyl or a ferrocenyl, said groups comprising possibly one or more substituents selected from among a halogen such as chlorine, fluorine or bromine, an —NO₂ group, a C₁₋₅ alkyl a C₁₋₅ alkoxy, a saturated or unsaturated C₁₋₇ cycloalkyl which can be fused or unfused, a polystyryl group, a fused or unfused aryl group which can be optionally substituted by a C₁₋₄ alkyl, a C₁₋₄ alkoxy or a halogen, said groups comprising possibly one or more heteroatoms such as O, N or Si,
as ligand in a process for enantioselective reduction of unsaturated compounds carrying functional groups by a hydrogen transfer method.

2. Use according to claim 1, characterized in that the optically active N-substituted chiral derivative of norephedrine responds to formula (II) below:
(II)
in which:
R1 and R2 have the same meaning as above and R3, R4, R5, R6 and R7, which can be identical or different, are selected from among a hydrogen atom, a halogen atom such as chlorine, fluorine or bromine, an —NO₂ group, a C₁₋₅ alkyl group, a C₁₋₅ alkcoxy group, a fused or unfused C₁₋₇ cycloalkyl group, a fused or unfused aryl group, possibly substituted by a C₁₋₅ alkyl, a C₁₋₅ alkoxy, a halogen, said groups comprising possibly one or more heteroatoms such as O, N or Si.

3. Use according to claim 1 or 2, characterized in that the optically active N-substituted chiral derivative of norephedrine responds to formula (I) or formula (II) in which:
R1 represents a hydrogen atom, a C₁₋₄ alkyl such as methyl, ethyl, propyl, isopropyl or a phenyl,
R2 represents a group selected from among a 2-furanyl, a 2-thiophenyl, a 3-thiophenyl, a ferrocenyl, an aryl of formula (III) below:
(III)
in which R8, R9, R10, R11 and R12, which can be identical or different, are selected from among a hydrogen atom, a halogen atom such as chlorine, fluorine or bromine, an —NO₂ group, a C₁₋₅ alkyl group, a C₁₋₅ alkoxy group, a fused or unfused C₁₋₇ cycloalkyl group, a fused or unfused aryl group, possibly substituted by a C₁₋₅ alkyl, a C₁₋₅ alkoxy, a halogen, said groups comprising possibly one or more heteroatoms such as O, N or Si.

4. Use according to any one of claims 1 to 3, characterized in that the optically active N-substituted chiral derivative of norephedrine responds to formula (IV) below:
(IV)
in which Ar is a phenyl group carrying one or more substituents such as a halogen, a hydrocarbon group which can be cyclical and/or acyclical, aliphatic and/or aromatic, comprising one or more carbon atoms, and possibly one or more heteroatoms such as O, N and Si, as well as one or more halogens such as F, Cl, Br or I.

5. Use according to any one of claims 1 to 4, characterized in that the optically active N-substituted chiral derivative of norephedrine responds to formula (V) below:
(V)
in which:
R1, R3, R4, R5, R6 and R7 have the same meaning as in formula (I),
R8, R9, R11 and R12 have the same meaning as in formula (III) and
R13, R14, R15, R16 and R17, which can be identical or different, are selected from among a hydrogen atom, a halogen such as chlorine, fluorine or bromine, an —NO₂ group, a C₁₋₅ alkyl, a C₁₋₅ alkoxy, a C₁₋₇ cycloalkyl, a polystyryl group, an aryl group possibly substituted by a C₁₋₄ alkyl, a C₁₋₄ alkoxy or a halogen, said alkyl, alkoxy, cycloalkyl, polystyryl, aryl groups comprising possibly one or more heteroatoms such as O, N or Si.

6. Use according to any one of claims 1 to 5, characterized in that the optically active N-substituted chiral derivative of norephedrine is selected from among:
(1S,2R)-N-(4-biphenylmethyl)-norephedrine,
(1S,2R)-N-(4-ethoxybenzyl)-norephedrine,
(1S,2R)-N-(4-ethylbenzyl)-norephedrine,
(1S,2R)-N-(2-chlorobenzyl)-norephedrine,
(1S,2R)-N-(2-methylbenzyl)-norephedrine,
(1S,2R)-N-(2,5-dimethylbenzyl)-norephedrine,
(1S,2R)-N-(1-naphthyl)-norephedrine,
(1S,2R)-N-(2-thiophenylmethyl)-norephedrine,
(1S,2R)-N-(1-thiophenylmethyl)-norephedrine,
(1S,2R)-N-(2-methoxybenzyl)-norephedrine,
(1S,2R)-N-(1-furanylmethyl)-norephedrine,
(1S,2R)-N-(4-ferrocenylmethyl)-norephedrine,
bis-(1S,2R)-N, N′-(1,1′-ferrocenyl)dimethyl)-norephedrine or their optical enantiomers.

7. Use according to any one of the previous claims, characterized in that the unsaturated compounds carrying functional groups are more specifically the carbonyls, imines, iminiums, oximes or derivatives comprising a double bond.

8. Use according to any one of the previous claims, characterized in that the optically active N-substituted chiral derivative of norephedrine responds to formula (VI) below:
(VI)
in which,
R18 is selected from among a C₁₋₅ alkyl, an aryl group, a heteroaryl group comprising one or more heteroatoms such as oxygen or nitrogen possibly substituted by a C₁₋₄ alkyl, by a C₁₋₄ alkoxy or by a halogen.
R19 is different from R18 and selected from among an oxyalkyl, an alkoxycarbonyl, an aryl possibly substituted by a C₁₋₄ alkyl, by a C₁₋₄ alkoxy or by a halogen, a heteroaryl₁ a heteroaryl comprising one or more heteroatoms such as oxygen or nitrogen possibly substituted by a C₁₋₄ alkyl, by a C₁₋₄ alkoxy or by a halogen, and
z represents an oxygen atom, a group of formula —NR20, —NOR20, —N(R20)₂Y or C(R20)₂ in which the R20 groups, which can be identical or different, represent a group selected from among a C₁₋₅ alkyl, an aryl group, a heteroaryl group comprising one or more heteroatoms such as oxygen or nitrogen possibly substituted by a C₁₋₄ alkyl, and Y is a counter anion such as an anionic organic or inorganic molecule.

9. Process for enantioselective reduction of unsaturated compounds carrying functional groups by a hydrogen transfer method, characterized in that an optically active N-substituted chiral derivative of norephedrine as defined in any one of claims 1 to 6 is brought to react with said unsaturated compound carrying functional groups in a basic or neutral medium in the presence of a catalytic quantity of a complex of a transition metal and a secondary alcohol as reducer.

10. Process according to claim 9, characterized in that the transition metal is iridium, rhodium or ruthenium.

11. Process according to either claim 9 or 10, characterized in that the complex of a transition metal is of the type [MCl₂(arene)]₂, in which M represents a transition metal such as rhodium, iridium or ruthenium, and arene means a compound of formula (VII) below:
(VII)
in which R21, R22, R23, R24, R25 and R26, which can be identical or different, are selected from among a hydrogen atom, a halogen, a C₁₋₅ alkyl group, an isoalkyl, a tertioalkyl, an alkoxy, with said alkyl and alkoxy groups comprising one or more heteroatoms such as O, N and Si.

12. Process according to one of claims 8 to 11, characterized in that the quantity of compound of formula (VI) in relation to the catalytic quantity of the complex of a transition metal is from 1 to 50,000, preferably from 10 to 10,000, and most preferably from 100 to 1000.

13. Process for enantioselective reduction of unsaturated compounds carrying functional groups, advantageously of formula (VI) as defined in claim 8, by a hydrogen transfer method, characterized in that it comprises employment of a catalytic quantity of a compound of formula (VIII) below:
(VIII)
in which: M and arene have the same meaning as in claim 11, and R, R1 and R2 have the same meaning as in claim 1, and R27 represents a halogen such as chlorine or bromine,
in a basic medium and in the presence of a secondary alcohol as reducer.

14. Process for enantioselective reduction of unsaturated compounds carrying functional groups, advantageously of formula (VI) as defined in claim 8, by a hydrogen transfer method, characterized in that it comprises employment of a catalytic quantity of a compound of formula (IX) or (X) below:
(IX)
(X)
in which: M, R, R1 R2 and arene have the same meaning as in formula (VIII) as defined in claim 13, and R29 and R28 each represent an electron pair,
in a neutral medium and in the presence of a secondary alcohol as reducer.

15. Process for enantioselective reduction of unsaturated compounds carrying functional groups by a hydrogen transfer method, characterized in that a catalytic quantity of a metallic complex of formula (IX) as defined in claim 13 is brought to react in the absence of base with said unsaturated compound carrying a functional group in the presence of a secondary alcohol as reducer.

16. Metallic complex that can be used in the process according to claim 13, characterized in that it responds to formula (XI) below:
(XI)
in which: M, R, R1, R2 and arene have the same meaning as in claim 13, R30 represents a hydrogen atom or an electron pair, R31 represents a hydrogen, a halogen such as chlorine or bromine, or an electron pair.
